(19) United States
(12) Patent Application Publication
(10) Pub. No.: US 2013/0184594 A1
(43) Pub. Date: Jul. 18, 2013

(54) Apparatus, Systems and Methods Analyzing Pressure and Volume Waveforms in the Vasculature

(71) Applicants: Kirk H. Shelley, New Haven, CT (US); David G. Silverman, West Redding, CT (US)

(72) Inventors: Kirk H. Shelley, New Haven, CT (US); David G. Silverman, West Redding, CT (US)

(73) Assignee: YALE UNIVERSITY, New Haven, CT (US)

(21) Appl. No.: 13/809,687

(22) PCT Filed: Jul. 12, 2011
(86) PCT No.: PCT/US2011/043701

§ 371 Date: Apr. 2, 2013

Related U.S. Application Data
(60) Provisional application No. 61/363,498, filed on Jul. 12, 2010.
(60) Provisional application No. 61/448,285, filed on Mar. 2, 2011.

(51) Int. Cl. A61B 5/0295; A61B 5/0205

(52) U.S. Cl. CPC A61B 5/0295; A61B 5/0205
USPC 600/484; 600/507; 600/506; 600/485; 600/486; 600/490

(57) ABSTRACT

Apparatus, systems and methods are provided for analyzing relative compliance in the peripheral vasculature. Such apparatus, systems and methods generally involve generating a plethysmograph (PG) signal, generating one or more pressure waveforms and comparing the pressure waveform(s) relative to the PG signal to determine compliance indexes associated particular regions of the vasculature. A relative compliance ratio may also be determined by comparing arterial and venous relative compliance indexes. Apparatus, systems and methods are also provided for analyzing a PG waveform. Such apparatus, systems and methods generally involve generating a plethysmograph (PG) signal and comparing amplitude modulation of the PG signal relative to baseline modulation of the PG signal to estimate a relationship between left ventricular end diastolic pressure and stroke volume. The estimated relationship may account for a phase offset for the time between when changes in venous return affect left ventricular end diastolic pressure and stroke volume.

### BACKGROUND

1. Technical Field

The present disclosure relates to apparatus, systems and methods for analyzing pressure and/or volume waveforms in the vasculature, e.g., in order to asses cardiac health and/or monitor relative compliance.

2. Background Art

The present disclosure expands on and extends the teachings of U.S. Pat. No. Publication No. 2007/0032732 to Shelley et al., entitled “Method of Assessing Blood Volume using Photoelectric Plethysmography” (referred to herein as the “Shelley Publication”). Accordingly, the foregoing patent publication is incorporated herein in its entirety.

Traditionally, invasive monitoring has been required to detect physiological factors such as decreases in intravascular volume. In recent years, however, intraoperative monitoring has been moving towards minimally-invasive or non-invasive techniques. This shift has been attributed to various considerations, including procedure time, cost, and known risks which for traditionally invasive techniques may include carotid artery puncture, arrhythmia, pneumothorax, and infection. Thus, pursuant to the need for minimally-invasive or non-invasive apparatus, systems and methods for assessing physiological factors, the Shelley Publication, disclosed, inter alia, various apparatus, systems and methods for non-invasivly monitoring changes in blood volume of a patient. Such information concerning relative blood volume is particularly valuable in the clinical setting. E.g., based on such information a clinician may more accurately administer diuretics and/or fluids, thereby preventing or counteracting conditions of hypervolemia, hypovolemia or dehydration.

Fluid status, however, is just one of several desirable physiological indicators. Other important indicators, include, e.g., vascular compliance and inotropy (cardiac strength). Thus, there remains a need for minimally-invasive or non-invasive apparatus, systems and methods for assessing physiological factors other than fluid status (such as vascular compliance and inotropy). Indicators of vascular compliance and inotropy may then be used to, inter alia, manage vasoconstrictors, vasodilators, inotropes, or other cardiovascular medications. This and other needs are addressed by the apparatus, systems and methods disclosed herein.

The Plethysmographic Waveform:

The pulse oximeter has rapidly become one of the most commonly used patient monitoring systems both in and out of the operating room. This popularity is undoubtedly due to the pulse oximeter's ability to non-invasively monitor peripheral oxygen saturation as well as basic cardiac functions (e.g., heart rate). In addition, pulse oximeters are relatively easy to use and comfortable for the patient.

While the predominant application of a pulse oximeter has been calculating oxygen saturation of Hb, a pulse oximeter also inherently functions as a plethysmograph (more particularly, a photoplethysmograph), measuring minute changes in blood volume in a vascular bed (e.g., finger, ear or forehead), i.e., based on changes in light absorption. See, e.g., Hertzman, A B, “The Blood Supply of Various Skin Areas as Estimated By the Photoelectric Plethysmograph,” Am. J. Physiol. 124: 328-340 (1938). Thus, the raw plethysmograph (PG) waveform is rich in information relevant to the physiology of the patient. Indeed, the PG waveform contains a complex mixture of the influences of arterial, venous, autonomic and respiratory systems on the peripheral circulation.

A typical pulse oximeter waveform presented to a clinician, however, is a highly filtered and processed specter of the raw PG waveform. Indeed, it is normal practice for equipment manufacturers to use both auto-centering and auto-gain routines on the displayed waveforms so as to minimize variations in the displayed signal. While such signal processing may benefit certain calculations, it often comes at the expense of valuable physiological data. Thus, the greater potential of the raw PG waveform, remains largely overlooked.

Even when the raw PG waveform is considered and analyzed, it is often oversimplified. Indeed, the PG waveform is typically characterized as comprising two components: (i) a “pulsatile” (AC) component (traditionally attributed to variations in blood volume caused by the cardiac pulse) and (ii) a “non-pulsatile” (DC) component (traditionally attributed to “static” blood volume in nonpulsatile tissue, such as fat, bone, muscle and venous blood). It has since been demonstrated that the DC component of the PG waveform is, in fact, not “non-pulsatile” but, rather, is “weakly-pulsatile.” It has further been demonstrated that a number of physiological factors impact both the AC and DC components and that the PG waveform is far more complex than originally suspected. Indeed, changes in venous blood volume often correspond to changes in end-diastolic volume (EDV), i.e., the volume of blood in the ventricles at the end of ventricular relaxation during diastole. More particularly, venous blood volume and venous compliance (e.g., relating to venous tone) affect venous blood pressure and the rate of venous return which in turn impact EDV. Thus, activation of the baroreceptor reflex, such as during acute hemorrhaging, causes venoconstriction which results in decreased venous compliance, improved venous return, and increased end-diastolic volume. Similarly, changes in arterial blood volume correspond to cardiac stroke volume, i.e., the difference between EDV and end-systolic volume (ESV). Cardiac output is determined as cardiac stroke volume multiplied by heart rate. Notably venous compliance is significantly (10-24 times) greater than arterial compliance.

Methods for extracting and analyzing the AC and DC components of the PG signal are provided in the Shelley publication. The ability to independently monitor changes in venous and arterial blood volume has many clinical applications. For example, changes in venous and arterial blood volume may be indicative of hypovolemia, e.g., due to bleeding, dehydration, etc. Decreased blood volume due to bleeding is, typically, characterized by an initial period of venous loss during which the cardiac output remains unaffected. With continued blood loss, decreased venous return eventually affects cardiac output (corresponding to arterial blood volume).

Since the main purpose of the pulse oximeter is determination of arterial oxygen saturation, most pulse oximeters filter out the venous (DC) component and normalize the arterial (AC) component to facilitate visualization of the signal. In addition, pulse oximeters are most commonly used on the finger, a region rich in sympathetic innervation that often reflects local (as opposed to systemic) alterations in vascular tone and volume status. See, e.g., Yamakage M, Itoh T, Iwasaki S, Jeong S-W, Namiki A, Can variation of pulse amplitude value measured by a pulse oximeter predict intravascular volume?, Anesthesiology 2004 abstracts; Dorlas J C, Nijiboer J A, Photo-electric plythysmography as a monitoring device in anaesthesia. Application and interpretations, BR J Anaethesia 1999 82(2):178-81; A245, H188, H236.

Peripheral Venous Pressure:

A further largely unexplored source of clinical information is pressure transduction of the standard intravenous line. A vast majority of hospitalized patients have a peripheral venous line. It is placed to allow fluids and medications to be given directly into the circulatory system. Until recently, the venous system's contribution to the circulatory system has been incorrectly identified as being insignificant. Indeed, veins do more than merely conduct blood to the heart; veins play a critical role in cardiovascular homeostasis. Thus, considering the ease of measurement from a peripheral venous catheter (PVC), further investigation of the utility and limitations of such a minimally invasive and inexpensive monitoring device is warranted.

Folkow, in the 1960s, studied the characteristics of veins and noted the huge disparity which existed in the literature concerning the amount of information on the arterial vs. the venous sides of the circulation. Folkow B, Mellander S., Veins and Venous Tone, Am Heart J. 1964; 68:397-408. Almost 50 years later, we have still not filled the gap. While arterial waveforms have been studied extensively, focus on the peripheral venous component has been scarce.

Controversy still exists concerning the role of peripheral veins and their contribution to the central volume in face of blood loss. Many studies in the late 1990s and early 2000s have shown a consistent correlation between peripheral venous pressure (PVP) and central venous pressure (CVP). See, e.g., Weingarten T N, Sprung J, Munis J R., Peripheral venous pressure as a measure of venous compliance during pheochromocytoma resection, Anesth Analg. 2004; 99:1035-7; and Charalambous C, Barker T A, Zipitis C S, Siddique I, Swindell R, Jackson R, et al., Comparison of peripheral and central venous pressures in critically Ill patients, Anaesth Intensive Care. 2003; 31:34-9. While CVP waveforms characteristically show a-, c-, and v-waves, PVP waveforms often appear as a more dampened sinusoidal pattern. Munis et al. reported mean PVP values of 13 mm Hg, CVP values of 10 mm Hg, with a PVP-CVP difference of 3 mm Hg (see Munis J. R., Bhatia et al., Peripheral venous pressure as hemodynamic variable in neurosurgical patients, Anesth Analg 2001; 91(1): 172-9). Amar et al. observed mean PVP values of 9 mm Hg and a mean CVP value of 8 mm Hg in 100 intraoperative patients (see Amar D, Melendez J A, Zhang H, Dobres C, Leung D H, Padilla R E, Correlation of peripheral venous pressure and central venous pressure in surgical patients, J Cardiothorac Vase Anesth. 2001; 15:40-3). Hadimioglu et al. came to the same conclusions in patients undergoing kidney transplant (see Hadimioglu N, Ertug Z, Yegin A, Sanli S, Gurkan A, Demirbas A, Correlation of peripheral venous pressure and central venous pressure in kidney recipients, Transplant Proc. 2006; 38:440-2). Baty et al studied 29 infants and children post cardiopulmonary bypass. The difference between peripheral venous pressure and central venous pressure in these patients was 11±3 mm Hg. No clinically significant variation in the accuracy of the technique was noted based on the actual CVP value, size of the PIV, its location, or the patient's weight (see Baty L, Russo P, Tobias J D, Measurement of central venous pressure from a peripheral intravenous catheter following cardiopulmonary bypass in infants and children with congenital heart disease, J Intensive Care Med. 2008; 23:136-42).

Other authors have done similar assessments in patients undergoing right hepatectomy. In Choi et al., a central venous catheter (CVC) was placed through the right internal jugular vein and a peripheral venous catheter (PVC) was inserted at the antecubital fossa in the right arm. A total of 1,430 simultaneous measurements of CVP and PVP were recorded. Choi concluded the difference between PVP and CVP was within clinically acceptable agreement and the degree of difference tended to remain relatively constant throughout the right hepatectomy in living donors. (See Choi S J, Gwak M S, Ko J S, Kim G S, Kim T H, Ahn H, et al., Can peripheral venous pressure be an alternative to central venous pressure during right hepatectomy in living donors?, Liver Transpl. 2007; 13:1414-21). Holtman et al. studied the correlation of both variables in patients undergoing liver transplant. The nature of the liver transplant surgery allowed the authors to test the durability of the PVP/CVP correlation during extreme derangements of physiology, including IVC crossclamp, brisk hemorrhage, and reperfusion of the donor graft. One unexpected finding, not previously reported in other studies, was the much weaker PVP/CVP correlation at low filling pressures. It was suggested that at low filling pressures, peripheral veins intermittently collapse, interrupting their continuity with the central circulation and thus leading to PVP/CVP divergence. (See Holtman N, Braunfeld M, Holtman G, Mahajan A., Peripheral venous pressure as a predictor of central venous pressure during orthotopic liver transplantation, J Clin Anesth. 2006; 18:251-5).

According to Munis et al. (2001), PVP may be used as an indirect measure of venous volume since pressure is related to volume/compliance. Alternatively, it was reported that fluctuations of PVP are highly influenced by changes in vascular tone. Thus, measurements of volume status using PVP may be distorted by local changes in vascular tone. Vincent at al. documented that hand vein compliance decreases in responses to the alpha-agonist phenylephrine. Vincent J, et al., Cardiovascular reactivity to phenylephrine and angiotensin II: comparison of direct venous and systemic vascular responses, Clin Pharmocol Ther 1992; 51:68-75.

Moreover, the relationship of peripheral venous pressure and central venous pressure differs among patients. For example, the offset in Munis' study averaged 3.0 mmHg, ranging from 0.5 to 8.9 mmHg over 15 subjects. Similarly, Pederson et al. reported a mean gradient of 2.6 cm H₂O and a range of 0.7 to 5.8 cm H₂O between the antecubital vein and right atrium. Hence, without a baseline comparison to CVP (which requires invasive insertion of a central venous catheter), it is difficult to determine the accuracy of PVP measurements.

Generally, while there have been attempts to relate PVP to CVP (see, e.g., Eustace B R., A comparison between peripheral and central venous pressure monitoring under clinical conditions, Injury 1970; 2(1):12-18; Choi S J, Gwak M S, Ko J S, Kim G S, Kim T H, Ahn H, et al., Can peripheral venous pressure be an alternative to central venous pressure during right hepatectomy in living donors?, Liver Transpl. 2007; 13:1414-21; Hoffman N, Braunfeld M, Holtman G, Mahajan A., Peripheral venous pressure as a predictor of central venous pressure during orthotopic liver transplantation, J Clin Anesth. 2006; 18:251-5; Milhoan K A, Levy D J, Shields N, Rothman A., Upper extremity peripheral venous pressure measurements accurately reflect pulmonary artery pressures in patients with cavopulmonary or Fontan connections, Pediatr Cardiol. 2004; 25:17-9.; Tobias J D, Johnson J O., Measurement of central venous pressure from a peripheral vein in infants and children, Pediatr Emerg Care. 2003; 19:428-30; and Desjardins R, Denault A Y, Belisle S, Carrier M, Babin D, Levesque S, et al., Can peripheral venous pressure be interchangeable with central venous pressure in patients undergoing cardiac surgery?, Intensive Care Med. 2004; 30:627-32), very little effort has been made to characterize the PVP waveform as an independent entity.

In the past, a number of investigators have advanced the concept that a small change in venous capacity, induced by venous constriction or relaxation, should markedly alter the cardiac output. See, e.g., Bartelstone H J., Role of the veins in venous return, Circ Res. 1960; 8:1059-76. In a delicately designed experiment involving dogs, Bartelstone was able to divide the venous system into two major components: (1) the central venous conduit, holding approximately 18% of the total blood volume and including the inferior Vena Cava (IVC) and the large vein continuations thereof; and (2) the reactive venous reservoir, containing approximately 45% of the total blood volume and including the veins between the capillaries and the central venous conduit. Bartelstone was also able to demonstrate that there exists an intravenous gradient which facilitates the movement from the reactive venous reservoir to the central venous conduit. Bartelstone further displayed that sympathetic stimulation had no significant impact on the central venous conduit, despite a dynamic impact on the reactive venous reservoir.

Venous Compliance:

Rothe in the 1990s effectively tackled the issue of compliance in the venous compartment. Thus, Rothe illustrated the concept of Mean Circulatory Filling Pressure (PMCF) described first by Guyton. He defined PMCF as mean vascular pressure that exists after circulatory arrest leading to redistribution of blood, so that all pressures are the same throughout the system. PMCF is thus related to the fullness of the circulatory system. This pressure has been measured and found to be close to 7 mm of Hg. This is clearly less than capillary pressure, but it is greater than the venous pressure at the atrio-caval junction under normal conditions. See Rothe C F, Mean circulatory filling pressure: its meaning and measurement, J Appl Physiol. 1993; 74:499-509.

As is evident from FIG. 1P, there is a huge contrast between venous and arterial compliance. The enormous compliance of veins allows for huge shifts of circulating volume in and out of the venous compartment. Peripheral venous constriction, as evidenced by the dashed line, tends to increase venous pressure and shift blood out of the venous compartment. Mohrman D, Cardiovascular Physiology. 6th ed. New York: McGraw-Hill Medical; 2006.

Two primary factors are known to affect peripheral venous tone: (1) blood volume within the veins: because the veins are so much more compliant, changes in circulating blood volume produce larger changes in the volume of blood in the veins than in any other vascular segment. Tyberg J V, How changes in venous capacitance modulate cardiac output, Pflugers Arch. 2002; 445:10-7; and (2) sympathetic venous activity. In addition, an increase in any force compressing veins from the outside has the same effect on the pressure inside veins as an increase in venous tone. Thus, such things as muscle exercise and wearing elastic stockings tend to elevate peripheral venous pressure.

The relationship between central venous pressure and venous return is known as the Venous Return Curve (see FIG. 2P). When venous tone changes, so does the central venous pressure. For example, whenever peripheral venous pressure is elevated by increases in blood volume or by sympathetic stimulation, the venous function curve shifts upward and to the right. Mohrman D 2006. This is believed to be caused by a decrease in venous capacitance which raises the mean circulatory pressure, which in turn tends to increase all intravascular pressures, and thus increases the preload of the heart. Id.

In the year 1955, Guyton, an investigator known for his valuable contributions to the field of physiology, explained the relationship between venous compliance and cardiac output. He used Starling's law for the determination of cardiac output which he defined as the relationship between the cardiac output and right atrial pressure and called the “cardiac response curve”. Guyton A C, Determination of cardiac output by equating venous return curves with cardiac response curves, Physiol Rev. 1955; 35:123-9

FIG. 3P demonstrates that peripheral venous constriction increases cardiac output by raising central venous pressure and moving the heart's function upward along a fixed cardiac function curve. FIG. 3P also depicts the response of the vasculature to hemorrhage into progressive steps (i.e., A to B to C to D) which does not happen discretely in reality. The actual course of a patient's net response to hemorrhage would appear to follow nearly a straight line from point A to point D.

The behavior of peripheral veins of the forearm, in response to hemorrhage or sympathetic activity, is conflicting. While Zoller was able to demonstrate that the forearm veins show intense venoconstriction in the absence of changes in other hemodynamic parameters, other studies have proved that those limb veins have very little role to play in contributing to the central blood volume. Zoller R P, Mark A L, Abboud F M, Schmid P G, Heistad D D, The role of low pressure baroreceptors in reflex vasoconstrictor responses in man, J Clin Invest. 1972; 51:2967-72.

Previous research has demonstrated the value of determining the vascular compliance to monitor alterations in peripheral vascular compliance. This can be done by plotting the volumetric information from the traditional strain gauge plethysmograph and the pressure information from arterial pressure monitors in the form of a pressure-volume graphic. See, e.g., Fitchett D, Bouthier J, Simon A, Levenson J, Safar M, Forearm arterial compliance: The validation of a plethysmographic technique for the measurement of arterial compliance, Clin Sci 1984; 67: 69-72; Westling H, Jansson L, Jonson B, Nilsen R, Vasoactive drugs and elastic properties of human arteries in vivo, with special reference to the action of nitroglycerine, Ear Heart J 1984; 5: 609-616; Fitchett D, Forearm arterial compliance: A new measure of arterial compliance, Cardiovasc Res 1984; 18: 651-656; Dahn I, Jonson B, A plethysmographic method for determination of flow and volume pulsation in a limb, J Appl Physiol 1970; 28: 333-336.

In Kirk H. Shelley, W. Bosseau Murray, David Chang, Arterial Pulse Oximetry Loops: A New Method of Monitoring Vascular Tone, Journal of Clinical Monitoring 1997; 13: 223-228, Shelley, et al. used a photoelectric plethysmograph signal supplied by a pulse oximeter as an indicator of volume changes and the pressure information from a radial artery pressure monitoring system to indicate “relative” compliance (since the plethysmographic signal is uncalibrated). Over the long term, however, it was concluded that this method was not very usefull, due in part to the non-specific nature of vascular compliance and in part to the multitude of factors that may influence vascular compliance, particularly in a peripheral vascular bed (e.g., in the finger). More particularly, the method was determined to only be a trend monitor which could detect increases or decreases in compliance but isolated measurements could not be used to guide clinical therapy.

### Ventilation-Induced Variation

It has been known for quite some time that ventilation, and especially positive pressure ventilation, can have a significant impact on the cardiovascular system. Cournand A, Modey H, Werko L & Richards D, Physiological studies of the effect of intermittent positive pressure breathing on cardiac output in man, Am J Physio 1948; 152:162-73; Morgan B, Crawford W & Guntheroth W, The homodynamic effects of changes in blood volume during intermittent positive pressure ventilation, Anesthesiology 1969; 30:297-305. The first formal studies of the effect of ventilator induced changes on arterial pressure were done in the early 1980's. Coyle J, Teplick R, Long M & Davison J. Respiratory variations in systemic arterial pressure as an indicator of volume status, Anesthesiology 1983; 59:A53; Jardin F, Fareot J, Gueret P et al., Cyclic changes in arterial pulse during respiratory support, Circulation 1983; 68:266-74. This recognition was soon followed by the intensive investigations of Azriel Perel who coined the term “systolic pressure variation” to describe this phenomenon. Along with various co-investigators, his research has encompassed over twenty articles and abstracts on the topic. From this significant body of work, based on both animal and human data, a number of conclusions have been drawn.

It has been shown that the responses of peripheral waveforms to respiration can be used as an indicator of hypovolemia. More specifically, arterial pressure waveforms in the periphery (e.g., radial artery) demonstrate increased systolic pressure variations in the context of hypovolemia (as a result of ventilation affecting venous return to the heart and hence affecting left ventricular stroke volume). The degree of systolic pressure variation and pulse pressure variation is a sensitive indicator of hypovolemia. Perel A, Pizov R & Cotev S, Systolic blood variation is a sensitive indicator of hypovolemia in ventilated dogs subjected to graded hemorrhage, Anesthesiology 1987; 67:498-502. This variation is significantly better than heart rate, central venous pressure and mean systemic blood pressure in predicting the degree of hemorrhage which has occurred. Perel A, Pizov R & Cotev S, Systolic blood pressure variation is a sensitive indicator of hypovolemia in ventilated dogs subjected to graded hemorrhage, Anesthesiology 1987; 67:498-502; Pizov R, Ya'ari Y & Perel A, Systolic pressure variation is greater during hemorrhage than during sodium nitroprusside-induced hypotension in ventilated dogs, Anesthesia & Analgesia 1988; 67:170-4. Chest wall compliance and tidal volume can influence systolic pressure variation. Szold A, Pizov R, Segal E & Perel A, The effect of tidal volume and intravascular volume state on systolic pressure variation in ventilated dogs, Intensive Care Medicine 1989; 15:368-71. Changes in systolic pressure variation correspond closely to changes in cardiac output. Ornstein E, Eidelman L, Drenger B et al., Systolic pressure variation predicts the response to acute blood loss, Journal of Clinical Anesthesia 1998; 10:137-40; Pizov R, Segal E, Kaplan L et al., The use of systolic pressure variation in hemodynamic monitoring during deliberate hypotension in spine surgery, Journal of Clinical Anesthesia 1990; 2:96-100.

Systolic pressure variation can be divided into two distinct components; Δup, which reflects an inspiratory augmentation of the cardiac output, and Δdown, which reflects a reduction in cardiac output due to a decrease in venous return. Perel A, Cardiovascular assessment by pressure waveform analysis, ASA Annual Refresher Course Lecture 1991:264. The unique value in systolic pressure variation lies in its ability to reflect the volume responsiveness of the left ventricle. Perel A, Cardiovascular assessment by pressure waveform analysis, ASA Annual Refresher Course Lecture 1991:264. In recent years, with the increased availability of the pulse oximeter waveform, similar observations have been made with this monitoring system. Partridge B L, Use of pulse oximetry as a noninvasive indicator of intravascular volume status, Journal of Clinical Monitoring 1987; 3:263-8; Lherm T, Chevalier T, Troche G et al., Correlation between plethysmography curve variation (dpleth) and pulmonary capillary wedge pressure (pcup) in mechanically ventilated patients, British Journal of Anesthesia 1995; Suppl. 1:41; Shamir M, Eidelman L A et al., Pulse oximetry plethysmographic waveform during changes in blood volume, British Journal Of Anaesthesia 82(2): 178-81 (1999).

To date, however, there has been remarkably little work done to document or quantify the phenomenon of systolic pressure variation. Limitations of the aforementioned include, inter alia, reliance on positive pressure and mechanical ventilation; and the requirement of ventilator maneuvers, such as periods of apnea.

As for detecting systolic pressure variation, it is noted that changes in intrathoracic pressure during ventilation causes variations in the PG signal. Fluctuations in the PG signal due to respiration/ventilation can be detected. See, e.g., Johansson A & Oberg P A, “Estimation of respiratory volumes from the photoplethysmographic sit. Parti: Experimental results,” Medical and Biological Engineering and Computing 37(1): 42-7 (1999). Respiratory-induced fluctuations have been used in the past in an attempt to estimate the degree of relative blood volume of patients undergoing surgery. See, e.g., Partridge B L, “Use of pulse oximetry as a noninvasive indicator of intravascular volume status,” Journal of Clinical Monitoring 3(4): 263-8 (1987); and Shamir M, Eidelman L A et al., “Pulse oximetry plethysmographic waveform during changes in blood volume,” British Journal of Anaesthesia 82(2): 178-81 (1999).

In the Shelley patent publication, it was first noted that respiration/ventilation modulates both AC and DC components of a PG waveform. Thus, the Shelley patent publication disclosed, inter alia, apparatus, systems and methods for monitoring changes in blood volume by separating the impact of respiration/ventilation on the venous and arterial systems. More particularly, by isolating the impact of respiration/ventilation on predominantly arterial (AC) and predominantly venous (DC) components of the PG waveform one is able to independently assess changes in blood volume in different regions of the vasculature (arterial and venous). As noted in the Shelley patent publication, the degree of respiratory-induced variation of the AC component of the PG waveform corresponds to modulation of arterial blood volume (more particularly, cardiac stroke volume). Similarly, as noted in the Shelley patent publication, the degree of respiratory-induced variation of the DC component of the PG waveform corresponds to venous blood volume.

One method suggested by the Shelley patent publication for extracting and analyzing impact of respiration/ventilation on the venous and arterial systems includes comparing tracings of the peaks and valleys of the PG waveform. Thus, respiratory-induced variation of the AC and DC components may be isolated, e.g., based on the amplitude and the average of the PG waveform, respectively.

AC and DC components of a PG waveform may also be isolated by applying active frequency filters during sampling (the signal from the photodetector may be time demultiplexed such that each frequency can be processed independently). Thus, e.g., frequencies below 0:45 Hz may be concentrated in the DC signal and frequencies above 0:45 Hz in the AC signal (note this is consistent with the interval between heart beats rarely exceeding 2 seconds).

Another method suggested by the Shelley patent publication for assessing changes in blood volume involves harmonic analysis, e.g., Fourier analysis, of the PG waveform. Harmonic analysis allows for the extraction of underlying signals that contribute to a complex waveform. As disclosed in the Shelley patent publication, harmonic analysis of the PG waveform principally involves a short-time Fourier transform of the PG waveform. In particular, the PG waveform may be converted to a numeric series of data points via analog to digital conversion, wherein the PG waveform is sampled at a predetermined frequency, e.g., 50 Hz, over a given time period, e.g., 60-90 seconds. A Fourier transform may then be performed on the data set in the digital buffer (note that the sampled PG waveform may also be multiplied by a windowing function, e.g., a Hamming window, to counter spectral leakage). The resultant data may further be expanded in logarithmic fashion, e.g., to account for the overwhelming signal strength of the cardiac frequencies relative to the ventilation frequencies. It is noted that while the Shelley patent publication discloses using joint time-frequency analysis, i.e., a spectrogram, as a preferred technique for viewing and analyzing spectral density estimation of the PG waveform, a spectrum for the PG waveform, as used herein, may be extrapolated therefrom for any discrete sampling period.

According to the Shelley patent publication, PG waveform analysis, such as described above, may be used to independently monitor changes in arterial and venous blood volume. For instance, respiratory induced variation of the AC component, represented in the frequency-domain as side-band modulation around the cardiac signal, is indicative of changes in blood volume severe enough to affect cardiac output. Similarly, increased respiratory-induced variation of the DC component of a PG waveform, represented in the frequency domain as an increase in signal strength at the respiratory frequency, is indicative of venous loss (it is noted however that decreased cardiac output may also, at times, contribute to changes in the respiratory signal). Thus, by monitoring side-band modulation of the cardiac signal, one is able detect changes in cardiac output and arterial blood volume. Similarly, by monitoring variations at the respiratory frequency, one is able to detect changes in venous blood volume.

Analysis of venous waveforms has indicated that, like arterial waveforms, they too exhibit respiratory variations and change in response to physiologic challenges. Brecher et al. examined the relationship of respiration on the intrathoracic (the central venous conduit) and extrathoracic veins (the reactive venous reservoir). Brecher et al. conducted experiments using both spontaneously breathing and mechanically ventilated dogs. Pressure recordings were obtained from the jugular vein, femoral artery, intrapleural space and right atrium. Brecher concluded the following for spontaneous breathing under normal volume status: (1) thoracic aspiration during inspiration causes increase in blood flow to the right atrium significantly due to the emptying of the extrathoracic veins into the central veins; (2) flow does not increase further once the collapsed state of extrathoracic veins has been reached; and (3) if inspiration is long and deep enough, flow may even drop slightly below its inspiratory maximum due to the exhaustion of the extrathoracic reservoir and the progressively increasing resistance offered by the partially collapsed extrathoracic veins. Brecher then studied the same relationship under conditions of hyper and hypovolemia and concluded that identical degrees of thoracic aspiration increase venous return only moderately in the hypovolemic state as compared to euvolemic state. Brecher further noted that the greater the hypovolemia, the shorter the duration and amount of the aspiratory flow augmentation and the earlier the onset of the collapsed stage. (See Brecher G A, Mixter G, Jr., Effect of respiratory movements on superior cava flow under normal and abnormal conditions, Am J. Physiol. 1953; 172:457-61).

Respiratory variations in the central venous waveform have been described before. The respiratory induced variation in central vein pressure also causes variations in arterial blood pressure (ABP), as described above, and in peripheral venous pressure (PVP). Valves in the venous system in the forearm may hinder hydrostatic continuity, implying that one single vein might not represent the entire venous system in the forearm. Whether the respiratory variation in PVP is a forward transmission of the change in arterial pressure or a backward transmission from the central venous system remains unclear. (see Nilsson, Macrocirculation is not the sole determinant of respiratory induced variations in the reflection mode, Physiological Measurement [0967-3334] 2003; 24:935).

### SUMMARY

Apparatus, systems and methods are provided according to the present disclosure for analyzing pressure and/or volume waveforms in the peripheral vasculature, e.g., in order to assess cardiac health and/or monitor relative compliance.

In exemplary embodiments, apparatus, systems and methods are provided for analyzing relative compliance in the peripheral vasculature. Such apparatus, systems and methods generally involve generating a plethysmograph (PG) signal, generating one or more pressure waveforms and comparing the one or more pressure waveform relative to the PG signal to determine one or more relative compliance indexes, wherein each of the one or more relative compliance indexes is associated with a particular region of the vasculature. Changes in one of the one or more relative compliance indexes advantageously reflects changes in compliance or impedance in the associated particular region of the vasculature. A relative compliance ratio may also be determined by comparing an arterial relative compliance index relative to a venous relative compliance index. The relative compliance ratio advantageously reflects relative compliance between arterial and venous regions of the vasculature. In exemplary embodiments, a relative compliance index may be determined by comparing a combined waveform (e.g., derived from arterial and venous pressure waveforms) relative to the PG signal, e.g., wherein corresponding arterial or venous components of the combined waveform and PG signal are compared. Alternatively a relative compliance index may be determined by individually comparing a pressure waveforms (e.g., an arterial or venous pressure waveform) relative to the PG signal. Thus, e.g., an arterial pressure waveform may be compared relative to an AC component of the PG signal and/or a venous pressure waveform may be compared relative to a DC component of the PG signal. In exemplary embodiments individually comparing the pressure waveform relative to the PG signal may include comparing corresponding arterial or venous components of the pressure waveform relative to the PG signal.

In exemplary embodiments, apparatus, systems and methods are provided for analyzing a PG waveform. Such apparatus, systems and methods generally involve generating a plethysmograph (PG) signal and comparing amplitude modulation of the PG signal relative to baseline modulation of the PG signal to estimate a relationship between left ventricular end diastolic pressure and stroke volume (also known as a Starling curve). The estimated relationship may advantageously account a phase offset between when changes in venous return affect left ventricular end diastolic pressure and when changes in venous return affect stroke volume. In exemplary embodiments the estimated relationship may advantageously be applied, e.g., to detect physiological conditions, to guide/titrate therapy, etc., e.g. be comparing a generated Starling curve relative to one or more known Starling curves.

Additional features, functions and benefits of the disclosed apparatus, systems and methods will be apparent from the description which follows, particularly when read in conjunction with the appended figures.

### BRIEF DESCRIPTION OF THE DRAWINGS

To assist those of ordinary skill in the art in making and using the disclosed apparatus, systems and methods, reference is made to the appended figures, wherein:

FIG. 1P depicts the relationship between volume and pressure both within the arterial and venous system.

FIG. 2P depicts the relation between venous filling pressure and venous return.

FIG. 3P depicts the relationship between the venous return curve and Starling cardiac output curve.

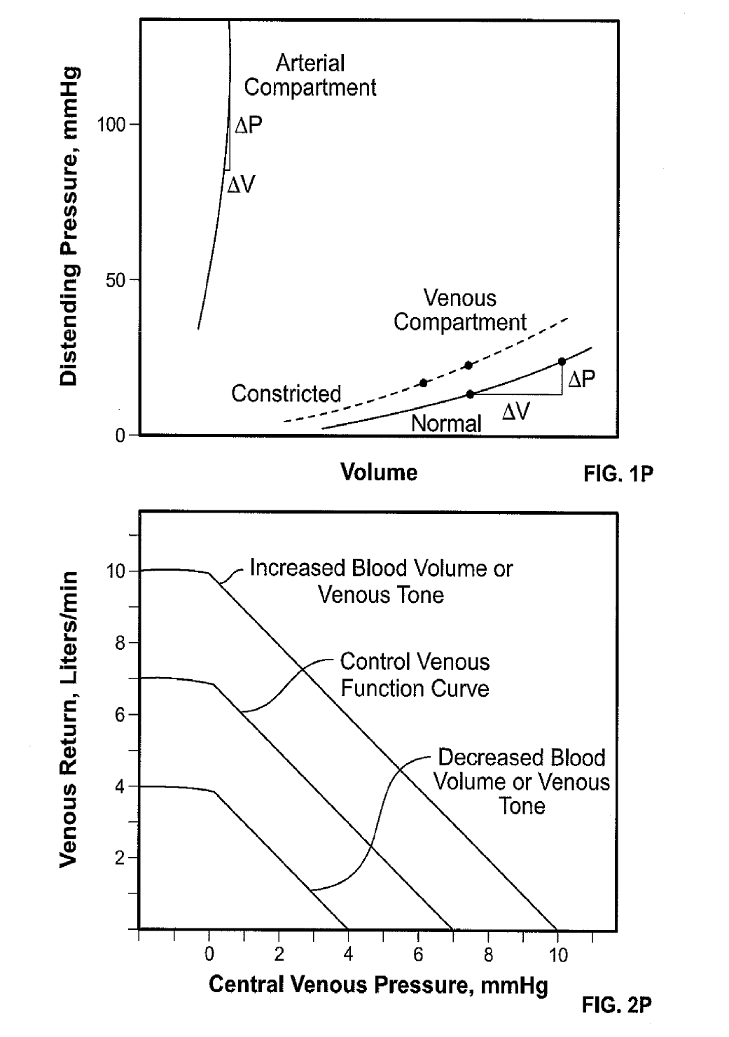

FIG. 1 depicts exemplary arterial and venous pressure waveforms, an exemplary respiratory waveform, an exemplary PG waveform and an exemplary combined arterial and venous pressure waveform, in the time domain, according to the present disclosure.

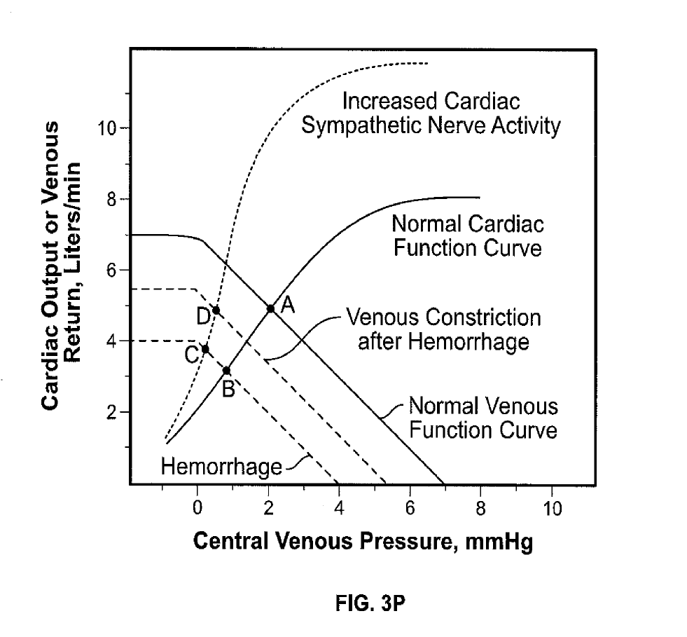

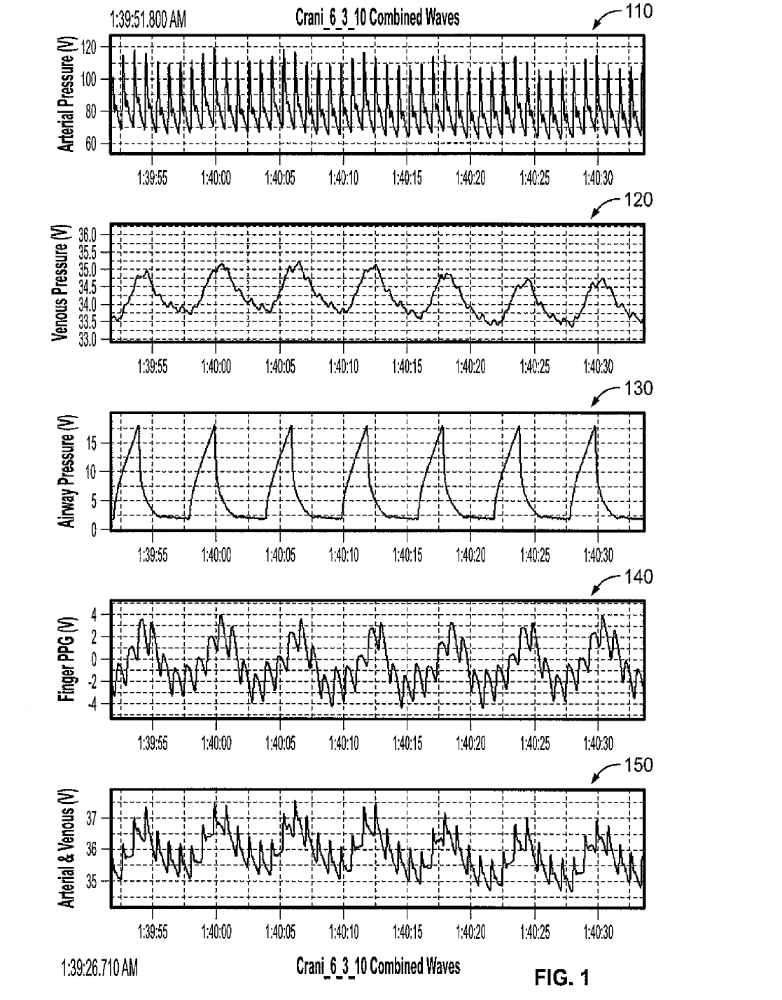

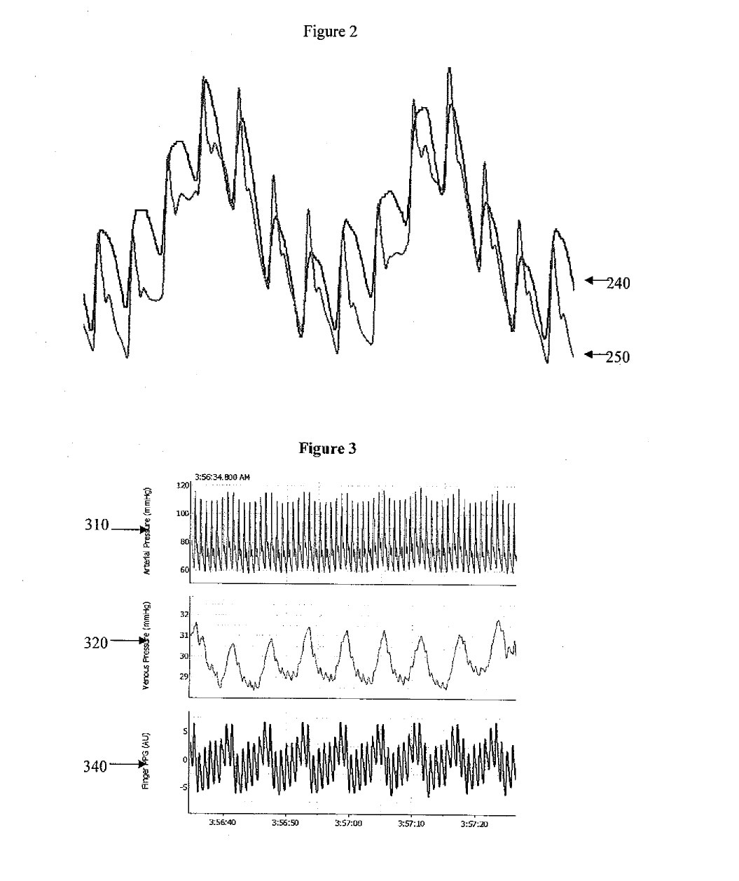

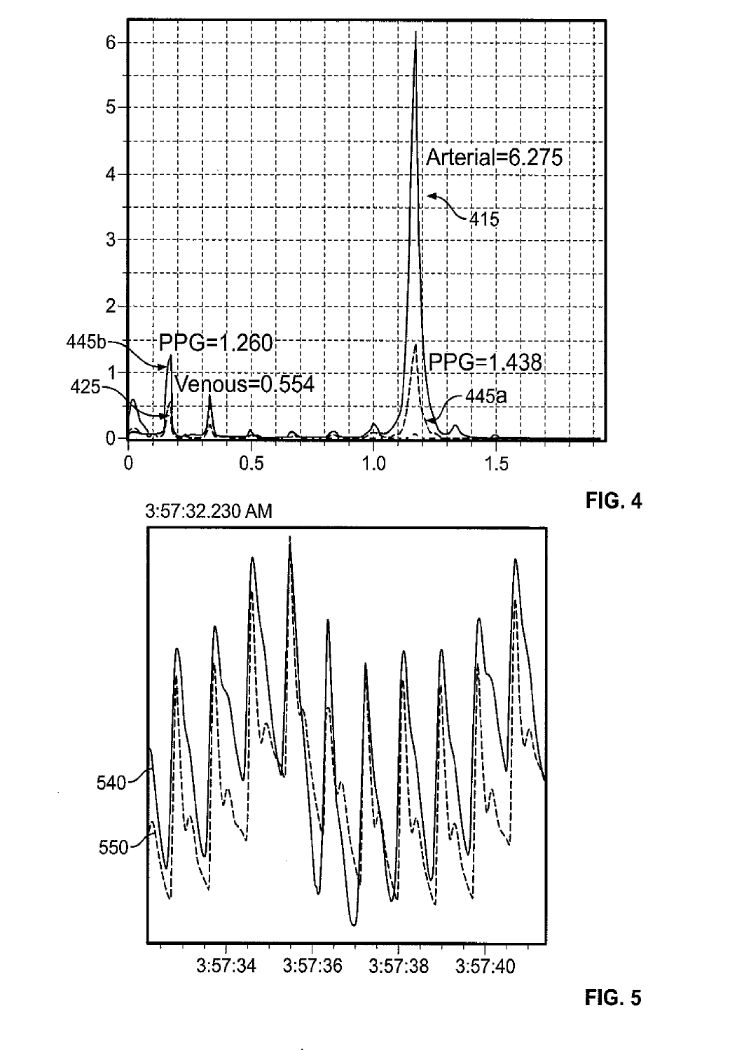

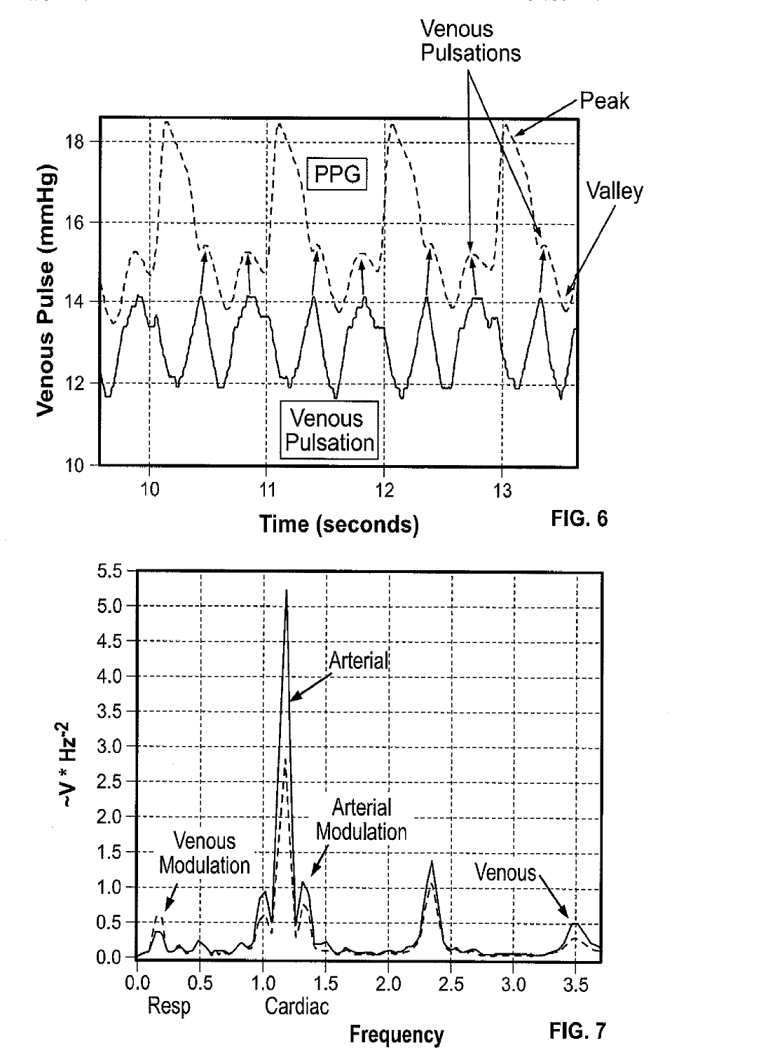

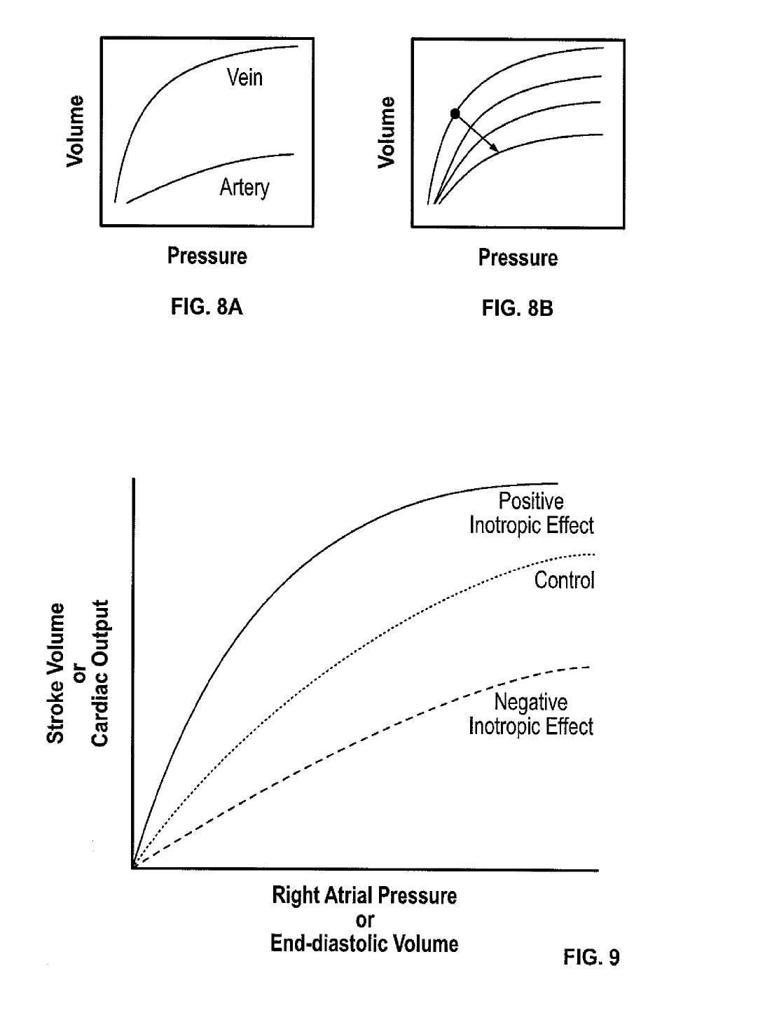

FIG. 2 depicts curve fitting an exemplary combined arterial and venous pressure waveform relative to an exemplary PG waveform, in the time domain, according to the present disclosure.

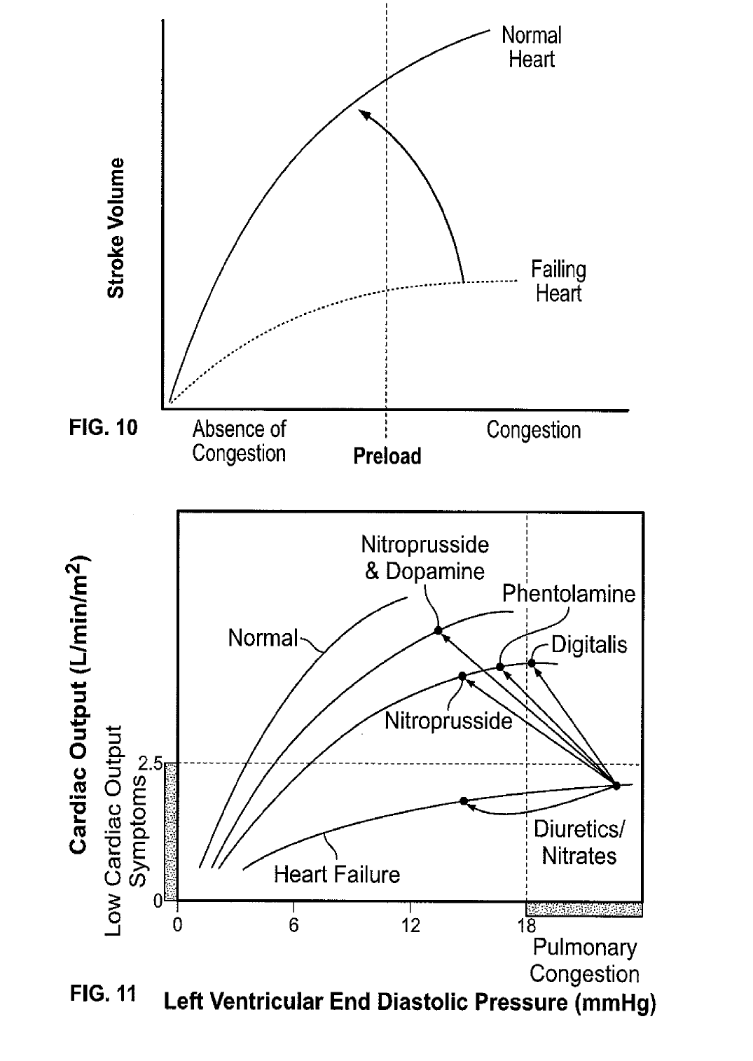

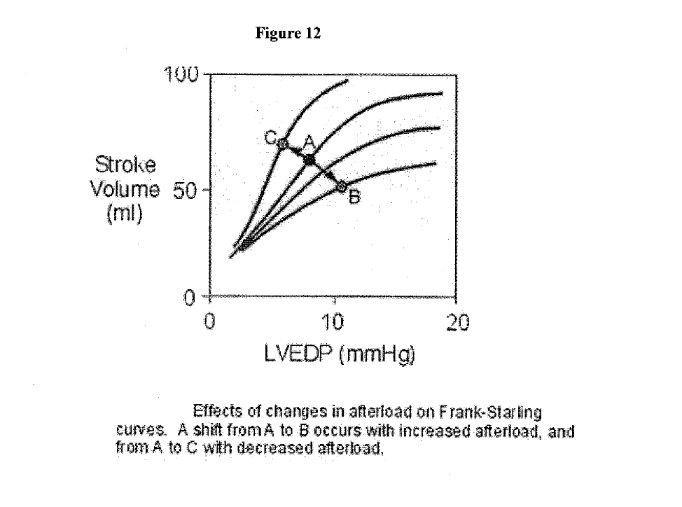

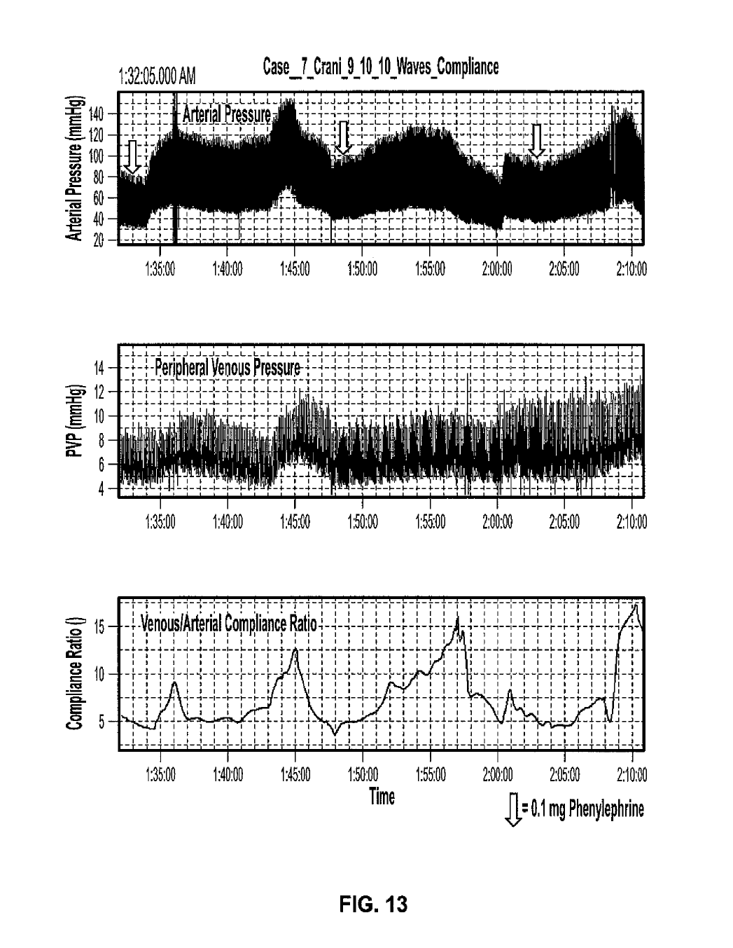

FIG. 3 depicts further exemplary arterial and venous pressure waveforms and a further exemplary PG waveform, in the time domain, according to the present disclosure.

FIG. 4 depicts the exemplary arterial and venous pressure waveforms and PG waveform, of FIG. 3, superimposed in the frequency domain, according to the present disclosure.

FIG. 5 depicts an exemplary best fit combination of the arterial and venous pressure waveforms of FIG. 3 relative to the exemplary PG waveform of FIG. 3, in the time domain, according to the present disclosure.

FIG. 6 depicts of an exemplary PG waveform overlaid with a venous pressure waveform, in the time domain, according to the present disclosure. Peaks, valleys and venous pulsations of the exemplary PG waveform are identified.

FIG. 7 depicts arterial and venous components of the PG signal as represented in the frequency domain, according to the present disclosure.

FIGS. 8a and 8b depicts exemplary compliance curves, according to the present disclosure.

FIGS. 9-12 depict exemplary starling curves related to inotropy, cardiac function, administration of medication, and compliance (afterload), respectively, according to the present disclosure.

FIG. 13 depicts the impact of vasopressor on a venous/arterial compliance ratio derived from arterial and venous pressure for a test subject, according to the present disclosure.

### DESCRIPTION OF EXEMPLARY EMBODIMENT(S)

According to the present disclosure, new and improved apparatus, systems and methods are provided for analyzing pressure and/or volume waveforms in the vasculature. In exemplary embodiments, the apparatus, systems and methods provided herein relate to analyzing pressure and volume waveforms in the vasculature. In further exemplary embodiments the apparatus, systems and methods provided herein relate to analyzing respiratory-induced variation (RIV) of waveforms in the peripheral vasculature. Note that as used herein, RIV is intended to encompass both spontaneous respiration and mechanical ventilation.

Apparatus Systems and Methods Comparing Pressure Waveforms to the PG Signal:

In exemplary embodiments, the apparatus, systems and methods may generally involve (i) generating a pressure waveform for a particular region of the vasculature, e.g., an arterial or venous pressure waveform, (ii) correlating the pressure waveform to a PG signal, and (iii) comparing the pressure waveform relative to the PG signal to determine a relative compliance index for the particular region of the vasculature, e.g., wherein changes in the relative compliance index are advantageously reflective of changes in compliance/impedance for the particular region of the vasculature (it is noted that relative compliance may be expressed as volume/pressure and relative impedance may be expressed as pressure/volume, wherein the relative compliance index may be indicative of both).

In exemplary embodiments, relative compliance indexes may be determined for each of arterial and venous regions of the vasculature (e.g., using arterial and venous pressure waveforms, respectively). A relative compliance ratio (e.g., venous compliance/arterial compliance, venous impedance/arterial impedance, arterial compliance/venous compliance, or arterial impedance/venous impedance) may then be determined by comparing the relative arterial compliance index relative to the relative venous compliance index, e.g., wherein the relative compliance ratio advantageously represents relative compliance between the arterial and venous regions of the vasculature. The relative compliance ratio could then be used to evaluate, e.g., if the patient's vasculature is too ‘tight’ or too ‘loose, and thereby facilitated administration of vasoconstrictors or vasodilators. Notably, relative compliance indexes may be separately determined, e.g., by individually comparing arterial and venous pressure waveforms to the PG signal, or simultaneously determined, i.e., by comparing a combined waveform derived from the arterial and venous pressure waveforms to the PG signal.

In general, an arterial pressure waveform may include any waveform/signal which is responsive to changes in arterial pressure and is correlatable to the PG signal, e.g., correlates to a component of the PG signal. In exemplary embodiments, the arterial pressure waveform may be generated using an arterial catheter a pulmonary artery catheter (PAC). There is growing evidence, however that invasive monitors of volume status, such as the PAC, may be a source of unacceptably frequent complications. Dalen J & Bone R, Is it time to pull the pulmonary artery catheter?, JAMA 1996; 276:916-14; Connors A, Speroff T & Dawson N, The effectiveness of right heart catheterization in the initial care of critically ill patients, JAMA 1996; 276:889-97. Thus, in exemplary embodiments, the arterial pressure waveform may be generated using other non-invasive or minimally invasive means, e.g., using a radial artery catheter, a finger arterial pressure monitor, a non-invasive blood pressure monitor such as a blood pressure cuff or the like. Continuous pressures also may be obtained by catheters in other vessels, such as brachial artery, femoral artery and aorta.

Similarly, a venous pressure waveform may include any waveform/signal which is responsive to changes in venous pressure and is correlatable to the PG signal. In exemplary embodiments, the venous pressure waveform may be generated using a central venous catheter (CVC) or other less invasive means, e.g., a peripheral venous catheter (PVC).

According to the present disclosure, arterial/venous pressure waveforms may substantially correlate to arterial/venous components of the PG waveform. More particularly, arterial and venous pressure waveforms may relate to venous and arterial components of the PG waveform by respective scaling factors, e.g., wherein the scaling factors represent relative compliance indexes for the arterial and venous pressure waveforms. Thus, e.g., an arterial pressure waveform, generated using a PAC may substantially correlate to an AC component of the PG waveform. More particularly, the arterial pressure waveform may relate to the AC component of the PG waveform by a scaling factor representative of a relative arterial compliance index. Similarly, e.g., a venous pressure waveform, generated using a CVC or PVC may substantially correlate to a DC component of the PG waveform and relate thereto by a scaling factor representative of a relative venous compliance index. Other arterial/venous pressure waveforms may also substantially correlate to components of the PG signal. For example, a pressure waveform reflective of systolic and/or diastolic blood pressure (BP) (e.g., generated using a non-invasive blood pressure monitor) may correlate to peaks and/or valleys of the PG signal, respectively.

As noted above, in exemplary embodiments, relative compliance indexes may be determined by comparing a combined waveform derived from the arterial and venous pressure waveforms to the PG signal. Thus, with reference to FIG. 1 an arterial pressure waveform 110 and a venous pressure waveform 120 are independently scaled and combined (combination waveform 150). The independent scale factors (representing the relative compliance indexes) are selected such that the combination waveform 150 best matches PG signal 140. Notably, the scaling of each of the arterial and venous pressure waveforms 110 and 120 may be relative to a same unit of measurement (although the unit of measurement may itself be arbitrary, since the PG signal 140 is typically uncalibrated). Thus, a relative compliance ratio may more accurately be determined. Please also note that a respiratory waveform 130 is depicted in FIG. 1 to demonstrate the occurrence of RIV in the other waveforms.

Referring to FIG. 2, an exemplary curve fitting technique is depicted. More particularly, a combination waveform 250 may be defined as:

(n*arterial pressure)+(m*venous pressure),

wherein, “n” and “m” represent relative compliance indexes for the arterial and venous pressure waveforms, respectively. Note that this formula may also be rewritten as:

x*((arterial pressure/y)+(venous pressure)),

wherein “x” is contingent on the arbitrary scaling of the PG signal and “y” represents a relative compliance ratio. According to the present disclosure, the combination waveform 250 may be compared to the PG signal 240, and the constants (“x” and “y” or “n” and “m”) selected, such that a best fit is achieved (e.g., using regression techniques; note that “best fit” may be defined based on root mean square error calculations).

Notably, as depicted in FIG. 2, the scale factor alone does not achieve a perfect fit. Indeed, for a healthy heart, one would expect the PG signal 240 to have greater RIV (amplitude and baseline) than the combination waveform 250. Thus, similar RIV's may be indicative a cardiac condition. It may therefore be beneficial to further compare the combination waveform 250 relative to the PG waveform 240, e.g., with respect to RIV.

As noted above, the apparatus systems and methods of the present disclosure are applicable both in the time and frequency domains. FIG. 3 depicts an arterial pressure waveform 310, a venous pressure waveforms 320 and a PG waveform 340. These waveforms are superimposed in the frequency domain representation of FIG. 4 (FFT power spectrum, 82 sec window, Hamming, 93.75% overlap, ˜15 min window of data). Based on the assumption that cardiac signal strength (˜0.8 Hz-2.5 Hz) is related primarily to the arterial system, a relative arterial compliance index may be calculated by comparing the cardiac signal strength 415 (e.g., peak signal strength, area under the curve, root-mean-square, etc.) for the arterial pressure waveform (or combined venous and arterial pressure waveforms) relative to the cardiac signal strength 445a for the PG waveform (e.g., PPG_{cardiac freq.}/Arterial pressure_{cardiac freq}). Similarly, based on the assumption that respiratory signal strength (˜0.1 Hz-0.4 Hz) is related primarily to the venous system, a relative venous compliance index may be calculated by comparing the respiratory signal strength 425 for the venous pressure waveform (or combined venous and arterial pressure waveforms) relative to the respiratory signal strength 445b for the PG waveform (e.g., PPG_{reap freq}. Venous pressure_{Resp freq}). Also, a relative compliance ratio (e.g., venous compliance/arterial compliance) may be determined as:

(PPG_{resp freq.}/Venous pressure_{Resp freq.})/(PPG_{cardiac freq.}/Arterial pressure_{cardiac freq.})

Thus, e.g., using FIG. 4, a relative compliance ratio (venous compliance/arterial compliance) may be determined as (1.260/0.554)41.438/6.275)=9.93. wherein (1.260/0.554) is the relative venous index and (1.438/6.275) is the relative arterial compliance indexes.

Plugging the relative venous and arterial compliance indexes into the formula ((n*arterial pressure)+(in*venous pressure) a combination waveform 550 may be derived (see FIG. 5). Notably, the combination waveform 550 is a pretty good fit relative to the PG signal (540).

In exemplary embodiments, PG values, venous pressure values, arterial pressure values, relative compliance indexes, and/or relative compliance ratios may be calibrated/normalized, such as with respect to cardiac signal strength, e.g., peak signal strength (in the frequency domain) or cardiac pulse amplitude, e.g., average pulse amplitude, (in the time domain). Thus, referring to FIG. 4, a normalized relative venous compliance index may be determined, e.g. as (e.g., PPG_{resp freq.}/PPG_{cardiac freq.})/Venous pressure_{Resp freq.}/Venous pressure_{cardiac freq.}).

Table 1, below, provides some of the possible correlations between components of the PG waveform and various pressure waveforms which may be used to determine relative compliance (see also FIGS. 6 and 7):

TABLE 1

| | Related | Frequency Domain | Exemplary Correlated Pressure |
|---|---|---|---|
| PG Component | Region | Comparison | Waveforms |
| Cardiac pulse | Arterial | Cardiac signal with | Cardiac pulse amplitude for arterial catheter |
| amplitude (e.g., | | amplitude modulation | waveform; cardiac pulse amplitude for |
| difference | | appearing as side | combined arterial and venous catheter |
| between peaks | | bands around the | waveform; or difference between systolic |
| and valleys) | | cardiac signal | and diastolic BP over a cardiac pulse. |
| includes RIV | | | |
| thereof) | | | |
| Base-line (e.g., | Venous | Respiratory signal | Venous catheter waveform; baseline for |
| average of peaks | | | combined venous and arterial catheter |
| and valleys; | | | waveform; or average of systolic and |
| includes RIV | | | diastolic BP over a cardiac pulse. |
| thereof) | | | |
| Peaks | Arterial | | Peaks for combined arterial and venous |
| | | | catheter waveform; or Systolic BP |
| Valleys | Venous | | Valleys for the combined arterial and |
| | | | venous catheter waveform; or Diastolic BP |
| Venous | Venous | Upper harmonics of | Venous Pulsations for the combined arteria |
| Pulsations | | cardiac signal | and venous catheter waveform |

According to the apparatus, systems and methods described herein it is now possible to calculate various indicia of relative compliance, e.g., relative arterial compliance indexes, relative venous compliance indexes, and relative compliance ratios which compare arterial and venous compliance. These indicia may advantageously facilitate monitoring cardiovascular events related to compliance as well as facilitate administration of compliance related medications, e.g., vasoconstrictors, vasodilators, etc., e.g., by comparing/plotting monitored indicia relative to standard venous and arterial compliance curves, such as depicted in FIG. 8. More particularly, FIG. 8a depicts exemplary venous and arterial compliance curves. Note, that the slope of the curves is equivalent to compliance. Thus, as depicted, venous compliance is roughly 10-20 times greater than arterial compliance at low pressures venous and roughly equal at higher pressures. FIG. 8b demonstrates how smooth muscle contractions decreases venous compliance (in the direction of the arrow).

In exeperiments conducted, 20 cardiac and 15 neurosurgical cases undergoing general anesthesia had their peripheral venous pressure (from a peripheral IV), arterial pressure (from radial artery) and PPG (from the finger of the same arm) waveforms collected via the GE S/5 Collect system. (It is noted that a standard blood pressure cuff reading could have been used instead of an a-line to measure arterial pressure in which case the arterial/venous compliance ratio could have been determined determined using only non-invasive or minimally invasive measures, e.g., blood pressure, a finger pulse oximeter waveform and a transduced peripheral IV. The waveforms were analyzed with LabChart7 using power spectrum, 82 sec Hamming window, 93.75% overlap. In each case, the venous/arterial compliance ratio was determined based on the following assumptions:
compliance=volume Δ/pressure Δ;
photoplethysmograph (PPG) modulation is a measure of volume change;
the arterial line and peripheral IV allows one to measure pressure change;
PPG modulation at the respiratory frequency (0.1 Hz-0.4 Hz)=movement of venous blood; and
PPG modulation at the cardiac frequency (0.8 Hz-2.5 Hz)=movement of arterial blood;

wherein, the venous/arterial compliance ratio=(PPG @ resp freq./venous pressure @ resp freq.)/(PPG @ cardiac freq./arterial pressure @ cardiac freq.)

Overall, the venous/arterial compliance ratio was observed to range aproximatly from 5 to 50 with hemodynamically stable patients ranging aproximatly from 10-25. Patients who were hemodynamically unstable, requiring intervention, tended to have lower ratios (e.g., <10). Doses of vasopressors (e.g. phenylephrine-0.1 mg) were observed to increase the ratio 2-3 fold. Notably the experimentally calculated compliance ratios were within the range of previously published ratios (See Klabunde, R., Cardiovascular physiology concepts. 2005, Philadelphia: Lippincott Williams & Wilkins).

With reference to FIG. 13, the impact of administering a vasopressor (0.1 mg phenylephrine; three (3) doses indicated by down arrows) on the peripheral venous/arterial compliance ratio for a hemodynamically unstable test subject is depicted. As depicted in FIG. 13, the hemodynamic instability of the patient is evidenced by the relatively low venous/arterial compliance ratio (approximately, 5) prior to each dose. For each dose, the venous/arterial compliance ratio can be seen to increase several fold, indicating the stabilizing effect of the vasopressor.

Apparatus Systems and Methods Analyzing RIV of the PG Waveform

Respiration and, in particular, positive pressure ventilation have a number of effects on the venous region of the vasculature. Positive pressure ventilation typically, introduces a force of approximately 30 mmHg with each breath. This force exceeds both venous pressure and pressure generated due to atrial contraction (the a-wave). Thus positive pressure pushes venous blood back to the peripheral vasculature resulting in markedly increased volume. Once positive pressure ends, those vessels empty very quickly, and blood flows into the heart.

Because it reverses blood flow, positive pressure markedly reduces venous return to the heart by blocking blood return from the periphery (although initially the ventilator may pump a little bit of blood flow into the heart). Decreased venous return has a delayed impact on left ventricular stroke volume and cardiac output. Namely, the effect of decreased venous return on the left side of the heart may be observed one or two beats after the blood is ejected from the right ventricle into the pulmonary circulation, left atrium and left ventricle (before being ejected as the left ventricular stroke volume). This is reflected in the AC component of the PG waveform as well as the upslope of an arterial pressure tracing. In exemplary embodiments, relative timing and phase relationships/synchrony of these events may be accounted for.

In exemplary embodiments, apparatus, systems and methods are provided for analyzing respiratory-induced variation (RIV) of the PG waveform in order to estimate a relationship between left ventricular end diastolic pressure (LVEDP) and stroke volume. This relationship is also known as a Starling curve. Note that LVEDP is related to the volume measure EDV. The ability to non-invasivly determine this relationship has broad clinical implications, e.g., with respect to monitoring inotropy, detecting cardiac failure, administering medication, and examining compliance (afterload) (see, FIGS. 9-12, respectively).

According to the present disclosure, a Starling curve may be generated based on the relationship between amplitude modulation (also referred to as RIV of the AC component) and baseline modulation (also referred to as RIV of the DC component) of a PG signal. More particularly, the RIV of the DC component is proportional to LVEDP. Likewise the RIV of the AC component is related to stroke volume.

As venous return (as may be measured by RIV of the DC component) changes, stroke volume (as may be measured by RIV of the AC component) should increase/decrease similar to a starling curve for a normal heart. A decreased response to changes in venous return could mean, e.g., that the patient has a weak heart or is overly hydrated. Thus, the relationship of RIV of the AC component relative to RIV of the DC component may, e.g., be used to identify disturbances of cardiac function.

The relationship may also be utilized to guide therapy. For example, if indications are that blood volume is low, then fluids can be added (this is similar to what was disclosed in the Shelley publication). If, however, volume appears normal (or high) and stroke volume appears low, then perhaps an inotrope is necessary to increase the strength of cardiac contractions.

The relationship of RIV of the AC component relative to RIV of the DC component may also be utilized to titrate therapy. For instance, during use of a vasodilator drug a decrease in blood pressure should have a favorable effect on the AC component of the PG waveform. The DC component, however, should also be monitored to make sure that the dilation is not creating a state of relative hypovolemia. Conversely, while the DC component may be used to optimize administration of a vasoconstrictive drug altered modulation of the DC component may indicate excessive vasoconstriction.

In determining the relationship between venous return and stroke volume it is important to account for an offset of a couple strokes between when an event affects venous return (right side of the heart) and when it affects stroke volume. Indeed, as noted above, whereas ventilation causes a direct effect on the right (venous) side of the heart, the effect on the left (arterial) side of the heart is indirect and modulated by factors such as changes in pre-ejection period and contractility. Thus, it may be favorable to incorporate a delay, e.g., with respect to the DC component, when comparing RIV of the AC component relative to RIV of the DC.

System Implementations:

It is explicitly contemplated that the disclosed systems and methods may be carried out, e.g., via a processing unit and/or system having appropriate software, firmware and/or hardware. As previously noted, a detection device may be used to obtain a waveform, e.g., a PG waveform or pressure waveform. Thus, in exemplary embodiments, the disclosed system may include an interface for communicating with an external processing unit, e.g., directly or over a network. The external processing unit may, for example, be a computer or other stand alone device having processing capabilities. Thus, in exemplary embodiments, the external processing unit may be a multifunction unit, e.g., with the ability to communicate with and process data for a plurality of measurement devices. Alternatively, the disclosed system may include an internal or otherwise dedicated processing unit, typically a microprocessor or suitable logic circuitry. A plurality of processing units may, likewise, be employed. Thus, in exemplary embodiments, both dedicated and external processing units may be used.

The processing unit(s) of the present disclosure generally include means, e.g., hardware, firmware and/or software, for carrying out one or more of the disclosed methods/processes of calibration/normalization. In exemplary embodiments, the hardware, firmware and/or software may be provided, e.g., as upgrade module(s) for use in conjunction with existing plethysmograph devices/processing units. Software/firmware may, e.g., advantageously include processable instructions, i.e., computer readable instructions, on a suitable storage medium for carrying out one or more of the disclosed methods/processes. Similarly, hardware may, e.g., include components and/or logic circuitry for carrying out one or more of the disclosed methods/processes.

A display and/or other feedback means may also be included/provided to convey detected/processed data. Thus, in exemplary embodiments, index values may be displayed, e.g., on a monitor. The display and/or other feedback means may be stand-alone or may be included as one or more components/modules of the processing unit(s) and/or system.

In general, it will be apparent to one of ordinary skill in the art that various embodiments described herein may be implemented in, or in association with, many different embodiments of software, firmware and/or hardware. The actual software code or specialized control hardware which may be used to implement the present embodiment(s) is not intended to limit the scope of such embodiment(s). For example, certain aspects of the embodiments described herein may be implemented in computer software using any suitable computer software language type such as, for example, C or C++ using, for example, conventional or object-oriented techniques. Such software may be stored on any type of suitable computer-readable medium or media such as, for example, a magnetic or optical storage medium. Thus, the operation and behavior of the embodiments may be described without specific reference to the actual software code or specialized hardware components. The absence of such specific references is feasible and appropriate because it is clearly understood that artisans of ordinary skill would be able to design software and control hardware to implement the various embodiments based on the description herein with only a reasonable effort and without undue experimentation.

Moreover, the systems and methods of the present disclosure may be executed by, or in operative association with, programmable equipment, such as computers and computer systems. Software that causes programmable equipment to execute the methods/processes may be stored in any storage device, such as, for example, a computer system (non-volatile) memory, an optical disk, magnetic tape, or magnetic disk. Furthermore, the disclosed methods/processes may be programmed when the computer system is manufactured or subsequently introduced, e.g., via a computer-readable medium.

It can also be appreciated that certain steps described herein may be performed using instructions stored on a computer-readable medium or media that direct a computer system to perform said steps. A computer-readable medium may include, for example, memory devices such as diskettes, compact discs of both read-only and read/write varieties, optical disk drives and hard disk drives. A computer-readable medium may also include memory storage that may be physical, virtual, permanent, temporary, semi-permanent and/or semi-temporary.

A “processor,” “processing unit,” “computer” or “computer system” may be, for example, a wireless or wireline variety of a microcomputer, minicomputer, server, mainframe, laptop, personal data assistant (PDA), wireless e-mail device (e.g., “BlackBerry” trade-designated devices), cellular phone, pager, processor, fax machine, scanner, or any other programmable device configured to transmit and receive data over a network. Computer systems disclosed herein may include memory for storing certain software applications used in obtaining, processing and communicating data. It can be appreciated that such memory may be internal or external to the disclosed embodiments. The memory may also include any means for storing software, including a hard disk, an optical disk, floppy disk, ROM (read only memory), RAM (random access memory), PROM (programmable ROM), EEPROM (electrically erasable PROM) and other computer-readable media.

Although the present disclosure has been described with reference to exemplary embodiments and implementations thereof, the disclosed systems, and methods are not limited to such exemplary embodiments/implementations. Rather, as will be readily apparent to persons skilled in the art from the description provided herein, the disclosed systems and methods are susceptible to modifications, alterations and enhancements without departing from the spirit or scope of the present disclosure. Accordingly, the present disclosure expressly encompasses such modification, alterations and enhancements within the scope hereof.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. A method for analyzing relative compliance in the peripheral vasculature, the method comprising:
generating a plethysmograph (PG) signal;
generating one or more pressure waveforms;
comparing the one or more pressure waveforms relative to the PG signal to determine one or more relative compliance indexes, wherein each of the one or more relative compliance indexes is associated with a particular region of the vasculature.

80. The method of claim 79, further comprising detecting one of (i) changes in compliance in the associated particular region of the vasculature, and (ii) changes impedance in the associated particular region of the vasculature, wherein changes in one of the one or more relative compliance indexes are reflective of the changes in compliance or impedance in the associated particular region of the vasculature.

81. The method of claim 79, wherein the one or more relative compliance indexes include an arterial relative compliance index associated with an arterial region of the vasculature, and a venous relative compliance index associated with a venous region of the vasculature, the method further comprising determining a relative compliance ratio by comparing the arterial relative compliance index relative to the venous relative compliance index, wherein the relative compliance ratio represents relative compliance between the arterial region of the vasculature and the venous region of the vasculature.

82. The method of claim 81, further comprising comparing the relative compliance ratio relative to standard venous and arterial compliance curves.

83. The method of claim 81, further comprising using the relative compliance ratio to monitor cardiovascular events.

84. The method of claim 79, further comprising, using the one or more relative compliance indexes to facilitate administration of vasoconstrictors or vasodilators.

85. The method of claim 79, wherein the one or more pressure waveforms include an arterial pressure waveform and a venous pressure waveform, wherein one or more of the one or more relative compliance indexes are determined by comparing a combined waveform derived from the arterial pressure waveform and the venous pressure waveform relative to the PG signal, and wherein the comparing the combined waveform relative to the PG signal includes comparing at least one of (i) corresponding arterial components of the combined waveform and PG signal, and (ii) corresponding venous components of the combined waveform and PG signal.

86. The method of claim 85, wherein the corresponding arterial components include one of (i) an AC component for each of the arterial pressure waveform and the PG signal, (ii) amplitude modulation of a cardiac pulse for each of the arterial pressure waveform and the PG signal, (iii) cardiac peaks for each of the arterial pressure waveform and the PG signal, (iv) a systolic component for each of the arterial pressure waveform and the PG signal, (v) a difference between systolic and diastolic components for each of the arterial pressure waveform and the PG signal, (vi) an average amplitude of a cardiac pulse for each of the arterial pressure waveform and the PG signal, (vii) a cardiac signal strength for each of the each of the arterial pressure waveform and the PG signal, and (viii) one or more sidebands around a cardiac signal for each of the arterial pressure waveform and the PG signal.

87. The method of claim 85, wherein the corresponding venous components include one of (i) a DC component for each of the venous pressure waveform and the PG signal, (ii) baseline modulation for each of the venous pressure waveform and the PG signal, (iii) cardiac valleys for each of the venous pressure waveform and the PG signal, (iv) venous pulsations for each of the venous pressure waveform and the PG signal, (v) a diastolic component for each of the venous pressure waveform and the PG signal, (vi) a respiratory signal strength for each of the each of the venous pressure waveform and the PG signal, and (vii) one or more upper harmonics of a cardiac signal for each of the venous pressure waveform and the PG signal.

88. The method of claim 79, wherein one or more of the one or more relative compliance indexes are determined by individually comparing one of the one or more pressure waveforms relative to the PG signal, wherein the one of the one or more pressure waveforms is an arterial pressure waveform responsive to changes in arterial pressure.

89. The method of claim 88, wherein the comparing the arterial pressure waveform relative to the PG signal includes comparing the arterial pressure waveform relative to an AC component of the PG signal.

90. The method of claim 88, wherein the comparing the arterial pressure waveform relative to the PG signal includes comparing corresponding arterial components of the arterial pressure waveform and the PG signal.

91. The method of claim 90, wherein the corresponding arterial components include one of (i) an AC component for each of the arterial pressure waveform and the PG signal, (ii) amplitude modulation of a cardiac pulse for each of the arterial pressure waveform and the PG signal, (iii) cardiac peaks for each of the arterial pressure waveform and the PG signal, (iv) a systolic component for each of the arterial pressure waveform and the PG signal, (v) a difference between systolic and diastolic components for each of the arterial pressure waveform and the PG signal, (vi) an average amplitude of a cardiac pulse for each of the arterial pressure waveform and the PG signal, (vii) a cardiac signal strength for each of the each of the arterial pressure waveform and the PG signal, and (viii) one or more sidebands around a cardiac signal for each of the arterial pressure waveform and the PG signal.

92. The method of claim 88, wherein the arterial pressure waveform is generated using one of (i) a pulmonary artery catheter, and (ii) a blood pressure cuff.

93. The method of claim 79, wherein one or more of the one or more relative compliance indexes are determined by individually comparing one of the one or more pressure waveforms relative to the PG signal, wherein the one of the one or more pressure waveforms is a venous pressure waveform, responsive to changes in venous pressure.

94. The method of claim 93, wherein the comparing the venous pressure waveform relative to the PG signal includes comparing the venous pressure waveform relative to a DC component of the PG signal.

95. The method of claim 93, wherein the comparing the venous pressure waveform relative to the PG signal includes comparing corresponding venous components of the venous pressure waveform and the PG signal.

96. The method of claim 95, wherein the corresponding venous components include one of (i) a DC component for each of the venous pressure waveform and the PG signal, (ii) baseline modulation for each of the venous pressure waveform and the PG signal, (iii) cardiac valleys for each of the venous pressure waveform and the PG signal, (iv) venous pulsations for each of the venous pressure waveform and the PG signal, (v) a diastolic component for each of the venous pressure waveform and the PG signal, (vi) a respiratory signal strength for each of the each of the venous pressure waveform and the PG signal, and (vii) one or more upper harmonics of a cardiac signal for each of the venous pressure waveform and the PG signal.

97. The method of claim 93, wherein the venous pressure waveform is generated using a central venous catheter or a peripheral venous catheter.

98. The method of claim 79, wherein the one or more pressure waveforms include a venous pressure waveform and an arterial pressure waveform, wherein the comparing the venous pressure waveform and the arterial pressure waveform relative to the PG signal includes determining relative scaling factors for the arterial and venous pressure waveforms such that a combined waveform derived from the relative scaling factors and the arterial and venous pressure waveform best matches the PG signal.

99. The method of claim 98, wherein the combined waveform is represented by one of (i) the formula (n*arterial pressure)+(m*venous pressure), wherein, n and m represent relative compliance indexes for the arterial and venous pressure waveforms, respectively, and (ii) the formula x*((arterial pressure/y)+(venous pressure)), wherein y represents a relative compliance ratio.

100. The method of claim 98, further comprising comparing the combined waveform relative to the PG signal and determining a cardiac condition, wherein comparing the combined waveform relative to the PG signal includes comparing respiratory-induced variations, wherein similar respiratory-induced variations are indicative of the cardiac condition.

101. The method of claim 79, wherein the one or more pressure waveforms include an arterial pressure waveform and a venous pressure waveforms, wherein a relative arterial compliance index is determined by comparing cardiac signal strength for the arterial pressure waveform relative to cardiac signal strength for the PG signal according to the formula:
relative arterial compliance=PPG_{cardiac freq.}/Arterial pressure_{cardiac freq.}
and wherein a relative venous compliance index is determined by comparing respiratory signal strength for the venous pressure waveform relative to respiratory signal strength for the PG signal according to the formula:
relative venous compliance=PPG_{resp freq.}/Venous pressure_{Resp freq.}

102. The method of claim 101, further comprising determining a relative compliance ratio according to the formula:
(PPG_{resp freq.}/Venous pressure_{Resp freq.})/(PPG_{cardiac freq.}/Arterial pressure_{cardiac freq.})

103. A system for analyzing relative compliance in the peripheral vasculature, the system comprising:
means for generating a plethysmograph (PG) signal;
means for generating one or more pressure waveforms; and
means for comparing the one or more pressure waveform relative to the PG signal to determine one or more relative compliance indexes, wherein each of the one or more relative compliance indexes is associated with a particular region of the vasculature.
